(19) United States
(12) Patent Application Publication
(10) Pub. No.: US 2010/0305204 A1
(43) Pub. Date: Dec. 2, 2010

(54) COMPOSITION USEFUL FOR THE PREVENTION OF ADVERSE EFFECT DUE TO THE USE OF PPAR-GAMMA AGONISTS

(75) Inventors: Menotti Calvani, Rome (IT); Stefania D'Iddio, Ciampino (IT); Paola Benatti, Grottaferrata (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Rome (IT)

(21) Appl. No.: 12/599,327

(22) PCT Filed: Apr. 28, 2008
(86) PCT No.: PCT/EP2008/055171

§ 371 Date: Feb. 2, 2010

(30) Foreign Application Priority Data
May 24, 2007 (EP) 07108801.7

(51) Int. Cl. A61K 31/221; A61P 43/00; A61P 7/10; A61P 19/10; A61P 3/04

(52) U.S. Cl. 514/547

(57) ABSTRACT

The present invention relates to the use of acetyl L-carnitine, or a pharmaceutically acceptable salt thereof, for the prevention of the adverse effects, such as osteoporosis, weight gain and edema, due to the use of PPAR-gamma agonists selected from the group consisting of spirolaxine or a glitazonederivative selected from the group consisting of roglitazone, pioglitazone androsiglitazone.

The present invention relates to the use of acetyl L-carnitine, or a salt thereof, for preparing a medicament for the prevention of the adverse effect due PPAR-gamma agonists.

### BACKGROUND OF THE INVENTION

PPAR-gamma agonists are agents useful for decreasing insulin resistance in pre-diabetic obese patients and for the treatment of type II diabetes.

Insulin resistance is a silent condition that increases the chances of developing type 2 diabetes. In insulin resistance condition the muscle, fat, and liver cells do not use insulin properly. The pancreas tries to keep up with the demand for insulin by producing more. Excess weight also contributes to insulin resistance because too much fat interferes with muscles' ability to use insulin. Lack of exercise further reduces muscles' ability to use insulin.

According to the American Diabetes Association, pre-diabetes can be defined as the state that occurs when a person's blood glucose levels are higher than normal but not high enough for a diagnosis of diabetes. About 11 percent of people with pre-diabetes, in the Diabetes Prevention Program standard or control group, developed type 2 diabetes each year during the average 3 years of follow-up. Other studies show that many people with pre-diabetes develop type 2 diabetes in 10 years.

Pre-diabetes was previously called Impaired Glucose Tolerance IGT and it has also been referred to as borderline or chemical diabetes.

Obesity which is linked to insulin resistance and pre-diabetes, can be an increased risk factor for hypertension, or high blood pressure which is one of the most important risk factors for cardiovascular disease, which can lead to a heart attack or stroke. If left untreated, hypertension can also lead to a wide variety of other life-threatening conditions, such as kidney damage and congestive heart failure.

People with obesity and insulin resistance are, therefore, at higher risk of cardiovascular disease. People with pre-diabetes have a 1.5-fold risk of cardiovascular disease compared to people with normal blood glucose, whereas people with diabetes have a 2- to 4-fold increased risk of cardiovascular disease. Always according to the American Diabetes Association people with pre-diabetes can delay or prevent the onset of type 2 diabetes. This can be done through lifestyle changes.

An estimated 20 million people have pre-diabetes in the U.S. and this number is growing rapidly. 50 Percent of the people who have pre-diabetes are likely to develop Type 2 diabetes.

Early diagnosis is important. In the early years of pre-diabetes or diabetes, the beta cells are progressively damaged by high blood sugars. Usually by the time diabetes is diagnosed, half of the beta cells are non-functional. This cannot be reversed so that the beta cells can go back to insulin production. However, when an early diagnosis of pre-diabetes is made, almost 100 percent of beta cells are functional. If lifestyle changes are made and some diabetes medications are used right away, many beta cells will stay healthy and make blood sugar control easier.

Diabetes or pre-diabetes can be detected and differentially diagnosed with one of the following tests:

Fasting Glucose Test, which measures blood glucose after not eating overnight. This test is most reliable when done in the morning. Fasting glucose levels of 100 to 125 mg/dL are above normal but not high enough to be called diabetes. This condition is called pre-diabetes or impaired fasting glucose (IFG), and it suggests that patient has probably had insulin resistance for some time. IFG is considered a pre-diabetic state, meaning that the patient is are more likely to develop diabetes but does not yet have it. Levels equal to or higher than 126 mg/dL are normally associated with diabetes.

Glucose Tolerance Test, which measures blood glucose after an overnight fast and 2 hours after patient drinks a sweet liquid provided by the doctor or laboratory. If patient blood glucose falls between 140 and 199 mg/dL, 2 hours after drinking the liquid, patient glucose tolerance is above normal but not high enough for diabetes. This condition, also a form of pre-diabetes, is called impaired glucose tolerance (IGT) and, like IFG, it points toward a history of insulin resistance and a risk for developing diabetes. Levels equal to or higher than 200 mg/dL are normally associated with diabetes.

Insulin resistance can be assessed with measurement of fasting insulin.

If conventional tests show that patient has IFG or IGT, the doctor may suggest changes in diet and exercise to reduce the risk of developing diabetes.

Diabetes is a widespread disease present throughout the world and is associated with major clinical complications including microvascular complications such as diabetic retinopathy, diabetic neuropathy and diabetic nephropathy, and macrovascular complications such as atherosclerosis, peripheral vasculopathies, obesity, hypertension myocardial infarction, stroke, polycystic ovary syndrome and syndrome X (J. Am. Osteopath. Assoc., 2000 October; 100(10):621-34; Jama, 2002 November, 27; 288(20):2579-88).

Said complications constitute a serious threat to the life and well-being of the individual.

The use of acetyl L-carnitine in the medical field is already known.

For example, WO 98/01128 discloses the use of the acetyl L-carnitine, isovaleryl L-carnitine, propionyl L-carnitine to increase the levels of IGF-1. Osteoporosis is included in the list of pathologies mentioned in WO 98/01128.

WO 98/41113 describes a therapeutic nutritive composition for patients with diabetes mellitus consisting of gamma linoleic acid, acetyl L-carnitine, mineral salts and vitamins.

U.S. Pat. No. 4,362,719 describes the use of the L-carnitine and the acyl L-carnitine in treating the juvenile onset diabetes mellitus.

U.S. Pat. No. 5,430,065 describes the use of the L-carnitine and the acyl L-carnitine in the long-term treatment of those patients with non insulin-dependent diabetes.

In Journal of Cellular Physiology 203; 2005; 439-446 is reported that the addition of acetyl L-carnitine to the culture medium dramatically affected the ability of myocytes to respond to insulin treatment.

None of the publication above mentioned describe that acetyl L-carnitine would have been useful for preparing a medicament for the prevention of the adverse effects due to PPAR-gamma agonists.

PPAR-gamma agonists, which include but are not limited to: spirolaxine and glitazone derivatives, are a known class of drugs used for preventing or reducing insulin resistance in obese patients and for the treatment of type II diabetes.

PPAR-gamma agonists acts as a transcriptional regulator of the genes linked to the glucose and lipid metabolism (Diabetes 47(4):507—Apr. 14, 1998). PPAR-gamma is expressed in a number of tissues raising the possibility that drugs that interact with it may induce clinical effects other than insulin sensitization. Prominent among the tissues in which PPAR-gamma is expressed is bone. In skeletal tissue, PPAR-gamma acts as a molecular switch that regulates the fate of pluripotent mesenchymal stem cells which have the ability to differentiate into adipocytes or osteoblasts. In vitro, PPAR-gamma agonists promote adipocyte differentiation in preference to osteoblast differentiation.

It is known in the art that anti-diabetic compounds PPAR-gamma, agonists are endowed with several adverse effects.

In fact, in J. Endocrinol. 2004 October; 183(1): 203-16 is reported that the use of Thiazolidinediones (TZDs) for the treatment of type 2 diabetes mellitus increases the risk of osteoporosis.

In Endocrinology, 2005 March; 146(3):1226-35; Epub 2004 December, is reported that peroxisome proliferator-activated receptor isoform gamma activation is a negative regulator of bone mass and suggest that the increased production of oxidized fatty acids with age may indeed be an important mechanism for age-related osteoporosis in humans.

The study published in the Journal of Clinical Endocrinology and Metabolism 2007; Jan. 30, found that short-term therapy with the commonly prescribed PPAR-gamma agonists inhibited bone formation and accelerated bone loss in a trial with 50 healthy postmenopausal women.

A study with more than 4,000 type 2 diabetes patients that unexpectedly found that the fracture rates, most commonly involving fractures of the homerous, hand and spine, were higher with rosiglitazone than with metformin or glibenclamide (glyburide) in women (Scrip No 3216, p 18).

In contrast, non-diabetic post-menopausal women with osteoporosis shown higher rate of fractures of femurs and lower limbs.

To date there is no explanations why the osteoporosis due to rosiglitazone causes most of the fractures in different part of the body respect to the osteoporosis in postmenopausal non-diabetic patients.

The presence of diabetes and the treatment with, for example, rosiglitazone characterize the differences between these two groups of patients.

Moreover, in The Annals of Pharmacotherapy 2001; January, Vol 35, is reported that patients treated with rosiglitazone shown a marked pulmonary and peripheral edema, and this may be a thiazolidinedione class effect.

In Journal of Clinical Endocrinology 86 Metabolism, Vol 86, n° 1, 2001 and Diabetes Care, Volume 24, N° 7, July 2001, is reported that patients treated with rosiglitazone shown an increased weight gain and fluid retention with oedema.

In the medical field there is still a perceived need the availability of compounds useful for reducing or preventing the adverse effect due to the use of PPAR-gamma agonists.

### DESCRIPTION OF THE INVENTION

It has now surprisingly been found that acetyl L-carnitine, or a pharmaceutically acceptable salt thereof, is useful for preparing a medicament for the prevention of the adverse effect due to the use of PPAR-gamma agonists. What is meant by pharmaceutically acceptable salt of acetyl L-carnitine is any salt of the latter with an acid that does not give rise to toxic or side effects.

These acids are well known to pharmacologists and to experts in pharmacy. Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

What is meant by pharmaceutically acceptable salt of L-carnitine is also a salt approved by the FDA and listed in the publication Int. J. of Pharm. 33 (1986), 201-217, which is incorporated herein by way of a reference.

It is therefore one object of the present invention the use of acetyl L-carnitine, or a pharmaceutically acceptable salt thereof, for preparing a medicament for the prevention of the adverse effect due to the use of PPAR-gamma agonists selected from the group consisting of spirolaxine of formula (I)

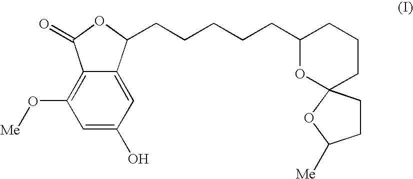

or a glitazone derivative selected from the group consisting of roglitazone, pioglitazone or rosiglitazone;

in which: said adverse effect due to the use of PPAR-gamma agonists is selected from the group consisting of osteoporosis, weight gain and edema.

It is a further object of the present invention a pharmaceutical composition comprising as active ingredients acetyl L-carnitine and a PPAR-gamma agonist, and optionally one or more diluents and/or excipients pharmaceutically acceptable.

### DETAILED DESCRIPTION OF THE INVENTION

The treatment of pre-diabetic obese patients shows an unexpected protective effect of the compound of the invention.

### Example 1

In a group of patients, treated orally for 12 months with rosiglitazone in a dose of 8 mg/day, six patients affected by osteoporosis with osteocalcine <3.5 pg/l and IGF1 <350 ng/ml were selected.

In these 6 patients, after a month of wash-out (no treatment with rosiglitazone) the serum levels of osteocalcine and IGF1 were lightly incremented even if not significantly (Table 1), while body weight (BW) and Total Body Water (TWB) didn't demonstrate variations (Table 2).

TABLE 1
Serum levels of osteocalcine (pg/L) and IGF1 (ng/ml) after treatment
with rosiglitazone (T0); and after 1-month wash-out (T1)

| | OSTEOCALCINE | | IGF1 | |
|---|---|---|---|---|
| Patient n° | T0 | T1 | T0 | T1 |
| 1 | 2.3 | 2.7 | 330 | 380 |
| 2 | 2.2 | 2.7 | 270 | 280 |
| 3 | 2.6 | 2.6 | 260 | 265 |
| 4 | 2.7 | 2.8 | 300 | 310 |
| 5 | 3 | 2.9 | 235 | 375 |
| 6 | 3.1 | 3 | 260 | 300 |
| Mean | 2.65 | 2.78 | 275.83 | 318.33 |
| ±Standard | 0.36 | 0.15 | 33.83 | 48.44 |
| Deviation | | | | |

| | | |
|---|---|---|
| P< (Student-t test) | NS | NS |

TABLE 2
BW and TBW (evaluated using a deuterated water system) after treatment
with rosiglitazone (T0); and after 1-month wash-out (T1).

| | |
|---|---|
| BW | TBW |
| Body weight | Total Body Water |

| Patient n° | T0 | T1 | T0 | T1 |
|---|---|---|---|---|
| 1 | 97.3 | 96.8 | 50.9 | 50.8 |
| 2 | 97.2 | 97 | 49.8 | 49.9 |
| 3 | 99 | 98.9 | 50.1 | 50 |
| 4 | 102.5 | 102 | 50.3 | 50.2 |
| 5 | 108.9 | 109 | 53.6 | 53.5 |
| 6 | 94.5 | 94.6 | 48 | 48 |
| Mean | 99.9 | 99.7 | 50.5 | 50.4 |
| ±Standard | 5.13 | 5.18 | 1.83 | 1.79 |
| Deviation | | | | |

| | | |
|---|---|---|
| P< | NS | NS |

After this wash-out period, a treatment with Rosiglitazone (at the same dose) in combination with acetyl L-carnitine (3 g/die per O.S.) was started.

After 4 months treatment with Rosiglitazone in combination with acetyl L-carnitine, serum levels of osteocalcine and IGF1 were monitored.

The results obtained are reported in Table 3 and 4, respectively.

TABLE 3
Serum levels of osteocalcine (pg/L) and IGF1 (ng/ml) at 1-
month rosiglitazone wash-out (T1) and after 4-months of treatment
with rosiglitazone and acetyl-L-carnitine (T2)

| | |
|---|---|
| OSTEOCALCINE | IGF1 |

| Patient n° | T1 | T2 | T1 | T2 |
|---|---|---|---|---|
| 1 | 2.7 | 3.9 | 380 | 530 |
| 2 | 2.7 | 3 | 280 | 480 |
| 3 | 2.6 | 3.7 | 265 | 470 |
| 4 | 2.8 | 3.9 | 310 | 470 |
| 5 | 2.9 | 3.1 | 375 | 440 |
| 6 | 3 | 3.3 | 300 | 480 |
| Mean | 2.78 | 3.48 | 318.33 | 478.33 |
| ±Standard | 0.15 | 0.40 | 48.44 | 29.27 |
| Deviation | | | | |

| | | |
|---|---|---|
| P< | p < 0.02 | p < 0.001 |

TABLE 4
Body Weight (BW) and Total Body Water (TBW) at 1-month
rosiglitazone wash-out (T1) and after 4-months of treatment
with rosiglitazone and acetyl-L-carnitine (T2)

| | |
|---|---|
| BW | TBW |
| Body weight | Total Body Water |

| Patient n° | T1 | T2 | T1 | T2 |
|---|---|---|---|---|
| 1 | 96.8 | 92.5 | 50.8 | 46.6 |
| 2 | 97 | 93.4 | 49.9 | 46 |
| 3 | 98.9 | 94.6 | 50 | 45.9 |
| 4 | 102 | 97.6 | 50.2 | 47.1 |
| 5 | 109 | 104.5 | 53.5 | 49.9 |
| 6 | 94.6 | 89.9 | 48 | 43.9 |
| Mean | 99.7 | 95.4 | 50.4 | 46.6 |
| ±Standard | 5.18 | 5.12 | 1.79 | 1.96 |
| Deviation | | | | |

| | | |
|---|---|---|
| P< | p < 0.001 | p < 0.001 |

The results reported in Table 3 shown that osteocalcine and

IGF1 significantly increased (p<0.02 and p<0.001).

The results reported in Table 4 shown that BW and TBW significantly decreased (p<0.001).

The results above reported shown that the compound of the invention is useful for treating the adverse effects due to the use of PPAR-gamma agonists.

The composition according to the present invention comprises active ingredients which are known in the medical sector and already used in clinical practice. Therefore, they are very easy to procure, inasmuch as they are products which have been on the market for some time and are of a grade suitable for human or animal administration.

Spirolaxine is a known compound described in EP 1368025 B1.

Glitazones are known compounds available on the market and can be prepared according to the methods described in the literature. Glitazones may be administered in an amount of from 1 mg to 10 mg/day, preferably 3 to 9 mg/day; most preferably 8 mg/day.

Acetyl L-carnitine is a known compound, the preparation process for which is described in U.S. Pat. No. 4,254,053. Acetyl L-carnitine may be administered in an amount of from 0.5 to 6 g/day, preferably 1 to 5 g/day; most preferably 3 g/day.

The daily dose to be administered, according to the present invention will depend on the judgement of the primary care physician, on the subject's weight, age and general conditions.

The composition of the invention can have a unitary form for simultaneous administration in which the active ingredients are present in a single pharmaceutical composition (tablet, sachet, capsule, vial) or the active ingredients can be administered in a coordinated sequential manner. In the latter case, the pharmaceutical composition can be formulated supplying the components in separate containers, accompanied by instructions for their sequential administration.

The compositions covered by the present invention are entirely conventional and are obtained with methods that are common practice in the pharmaceutical industry. According to the administration route opted for, the compositions will be in solid or liquid form, suitable for oral, parenteral or intravenous administration. The compositions according to the present invention contain, along with the active ingredient, at least one pharmaceutically acceptable vehicle or excipient. Particularly useful may be formulation adjuvants such as, for example, solubilising agents, dispersing agents, suspension agents and emulsifying agents. A general reference work is Remington's Pharmaceutical Sciences Handbook, latest edition.

1. (canceled)

2. The method according to claim 10 wherein the PPAR-gamma agonist is selected from the group consisting of spirolaxine of formula (I) 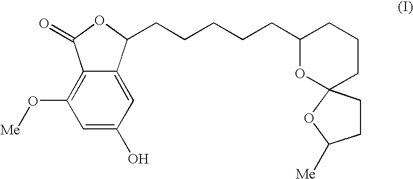
and a glitazone derivative selected from the group consisting of roglitazone, pioglitazone and rosiglitazone.

3. The method according to claim 10 wherein the adverse effect is selected from the group consisting of osteoporosis, weight gain and edema.

4. The method according to claim 10 wherein the pharmaceutically acceptable salt of acetyl L-carnitine is selected from the group consisting of chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichioroacetate, and trifluoroacetate.

5. Pharmaceutical composition comprising as active ingredients acetyl L-carnitine and a PPAR-gamma agonist, and optionally one or more diluents and/or excipients pharmaceutically acceptable.

6. Pharmaceutical composition of claim 5, wherein the PPAR-gamma agonist is selected from the group consisting of spirolaxine of formula (I) and a glitazone derivative selected from the group consisting of roglitazone, pioglitazone and rosiglitazone.

7. Pharmaceutical composition of claim 5, wherein the active ingredients are in solid or liquid form, suitable for oral or parenteral administration in the form of tablet, sachet, capsule or vial.

8. Pharmaceutical composition of claim 5, wherein the active ingredients are in a single pharmaceutical form for simultaneous administration.

9. Pharmaceutical composition of claim 5, wherein the active ingredients are in separate containers and administered in a coordinated sequential manner.

10. A method of preventing adverse effects due to the use of PPAR-gamma agonist in a subject comprising
administering an effective amount of acetyl L-carnitine, or a pharmaceutically acceptable salt thereof to said subject.

11. The method of claim 10, wherein said acetyl L-carnitine is administered in an amount of from about 0.5 to about 6 g/day.

12. The method of claim 10, wherein said acetyl L-carnitine is administered in an amount of from about 1 to about 5 g/day.

13. The method of claim 10, wherein said acetyl L-carnitine is administered in an amount of about 3 g/day.
